(19) United States
(12) Patent Application Publication
(10) Pub. No.: US 2013/0256171 A1
(43) Pub. Date: Oct. 3, 2013

(54) PACKAGING UNIT WITH FOLDED ENCLOSURE

(71) Applicants: Andrew Kerdemelidis, London (GB); Patrick John Foreman, London (GB)

(72) Inventors: Andrew Kerdemelidis, London (GB); Patrick John Foreman, London (GB)

(21) Appl. No.: 13/433,383

(22) Filed: Mar. 29, 2012

(51) Int. Cl. A61F 15/00; B65D 75/20

(52) U.S. Cl. 206/441; 206/476

(57) ABSTRACT

The invention relates to an improved hygienic packaging unit including a strip being arranged in a v-folded assembly, such strip having a sealed enclosure having a protective upper and lower layers wherein it is possible to separate the layers and expose the contents of the enclosure in a single fluid movement. In addition, the described packaging unit allows the sealed enclosure to be opened by using only one hand. Preferably, a plurality of packaging units are connected end-to-end and stacked in a z-folded assembly within a dispenser which facilitates the release of individual packaging units.

### FIELD OF THE INVENTION

The present invention relates generally to a packaging unit having a strip with folded enclosure.

More particularly, the invention relates to an improved hygienic packaging unit including a strip being arranged in a v-folded assembly, such strip having a sealed enclosure having a protective upper and lower layers wherein it is possible to separate the layers and expose the contents of the enclosure in a single fluid movement. In addition, the described packaging unit allows the sealed enclosure to be opened by using only one hand. Preferably, a plurality of packaging units are connected end-to-end and stacked in a z-folded assembly within a dispenser which facilitates the release of individual packaging units.

### BACKGROUND OF THE INVENTION

In general, adhesive dressings are constructed with two short protective tabs attached to the adhesive surface and with two free ends loosely covered by a non-adhesive dressing and then sealed within a pouch made of two external sheets. The ends of the two external packaging sheets protrude slightly, allowing them to be grasped (e.g. with two hands) and pulled apart to open up the packaging.

A disadvantage with such prior art conventional packaging and adhesive dressings is that they can be difficult to open. The protruding ends of the packaging are usually short and both face in the same direction. The user needs to locate the protruding ends, separate them and bend one or both backwards, grip one in each hand and pull them apart to access the dressing.

A second disadvantage with such prior art is that once the packaging is opened, the user is required to remove at least one of the protective tabs covering the dressing to expose one half of the adhesive part of the plaster. To do this a user must to hold down the plaster at one end with one hand and lift the free end of the protective tab with the other.

A third disadvantage with such prior art is that because the free end of the protective tabs cover the dressing part of the plaster there is a risk of cross-contamination of the dressing from any dirt or bacteria on the user's fingers when they lift the tab to remove it.

A fourth disadvantage with such prior art is that once the dressing and one end of the plaster are exposed it can be difficult to apply if the wound is on the user's hand or they only have one hand free. Further, once released, there is no means of simply pressing the wound to the plaster other than by holding the adhesive portion of the plaster itself which can reduce the tactile adhesion of the plaster.

A fifth disadvantage with such prior art is that because the plaster is not attached to a packaging strip in any way it is easy to drop when opening the packaging, which again risks cross-contamination of the dressing.

Various delivery systems have been described that assist the user in removing an individual sterile device from its package with only one hand. The following patents describe delivery systems that dispense the sterile device, requiring only one hand by the user: Haber, U.S. Pat. No. 4,733,797, Goldstein, U.S. Pat. No. 4,807,753, Moshel, U.S. Pat. No. 3,520,403, and Cooper U.S. Pat. No. 1,827,354. Although these disclosed delivery systems dispense the sterile device, requiring only one hand, the user must use two hands in the later application of the sterile device. Hence, there is a need for a packaged sterile device that may be dispensed and applied with only one hand.

Taulbee, deceased et al., in U.S. Pat. No. 4,993,586, and Adams IV, U.S. Pat. No. 3,835,992, both disclose an adhesive bandage dispensing package. Taulbee discloses an adhesive bandage sandwiched between an upper and lower protective strip. The adhesive side of the bandage is mounted facing downward on the lower protective strip. One end of the adhesive strip preferably attaches to a mounting pad. The mounting pad facilitates removal of the bandage from the lower protective strip, however, two hands are required to remove the mounting pad from the adhesive strip, and a portion of the sterile bandage must be contacted by the user. Therefore, a need exists for a sterile device, such as a bandage that may be dispensed and applied with one hand without contaminating any portion of the sterile device. Adams, IV, discloses a bandage dispensing package similar to Taulbee et al., which dispenses a bandage from a continuous roll. A mounting pad similar to that described by Taulbee et al. separates one end of the adhesive strips from the lower protective strip. In use, the user must use two hands to separate the mounting pad from the adhesive strip.

Heinecke et al. in U.S. Pat. No. 5,160,315 discloses a combined adhesive strip and transparent dressing delivery system. As disclosed in the specification, the adhesive strip is applied to the outside of a carrier. The adhesive strip is removed from the carriers and then is later applied to a transparent backing Similar to the prior art devices, the Heinecke et al. system requires two hands for the removal and application. Hence, there is a need for a bandage that may be dispensed and applied with only one hand.

Gilman in U.S. Pat. Nos. 5,018,516 and 4,600,001 discloses a wound dressing sandwiched between upper and lower layers of film. Although Gilman in the ‘001 patent describes his invention as providing “for sterility of the wound dressing layer during application”, the wound dressing, lower and upper layers must be packaged in an additional enclosure to maintain sterility. The additional enclosure may increase manufacturing costs, is cumbersome, and requires additional prep time to remove the packaged wound dressing from the additional enclosure. In addition, the removal from the enclosure and application of the wound dressing requires two hands. Hence, a need exists for a sterile delivery system enclosing a sterile device in a single enclosure, wherein the sterile device may be dispensed and applied with only one hand.

U.S. Pat. No. 5,891,078 discloses a dispenser allowing release of a sterile bandage with one hand and applied using one hand. However, the application of the bandage is cumbersome and requires a multi-layered flexible strip with a design that increases manufacturing costs.

Dotta in U.S. Pat. Nos. 4,235,337 and 6,719,137 discloses a “Rapidly opening sealed package for wound dressing adhesive tape” and a “Sealed package for adhesive wound dressing and apparatus therefore”. In both disclosures, the packaging is not designed to be opened with one hand, in particular, the packaging opens after the application of force in opposite directions on both ends of the external packaging. In addition, after opening, the external packaging is not attached underneath the adhesive strip meaning that it would be more difficult to present to the wound and apply accurately with one hand. Lastly, the package design is more difficult to manufacture by including two outer sheaths and the opening of the packing necessitates the removal of any protective layers over the adhesive strip.

Lauritzen in U.S. Pat. No. 4,545,372 describes a unitary adhesive bandage and package whereby the adhesive bandage and integral wrapper is constructed with a continuous length of bulky nonwoven bandage and wrapper material. There are significant disadvantages with using the same material for the outer packaging as the plaster. For example, the disclosed invention cannot be applied to breathable plasters as this would compromise the integrity of the seal. In addition, such a system would be much bulkier than using lightweight material specifically for packaging. Finally, using the same material to make the same material function as a plaster and packaging requires complex manufacturing techniques, applying different adhesives at precise points and inevitably means a compromise of optimal performance as either plaster, packaging or both.

There is a need for an apparatus and method that alleviates these problems in the prior art. The present invention overcomes these and other disadvantages.

### SUMMARY OF THE INVENTION

The present invention relates to a packaging unit that allows the release of contents sealed within a folded enclosure in a material strip.

In a first aspect the invention provides a packaging unit comprising:

a material strip folded to form an enclosure, such enclosure being sealed substantially along the edges of the material strip to form an upper and lower layer of said enclosure wherein said upper and lower layers can be separated upon the application of pulling force on either end of the material strip adjacent to said enclosure.

Preferably, the material strip having a v-folded edge substantially in its centre to form an enclosure;

an upper layer and lower layer of said enclosure, wherein said upper and lower layers are substantially parallel to each other;

proximal and distal tabs of material strip which are formed by folding back said upper and lower layers at the top enclosure edge of said enclosure, respectively;

wherein said enclosure is sealed along said top enclosure edge and its side enclosure edges;

wherein said upper and lower layers of said enclosure can be separated by pulling on the proximal and/or distal tab of said material strip;

whereby said enclosure is opened to dispense its contents.

Preferably, the content of said enclosure is an adhesive bandage although any other item having characteristics which allow stacked arrangement of the packaging units can be substituted.

Preferably, the proximal end of the strip is secured to the adjacent packaging unit with a strength greater than the adhesive strength of the inner edges of said enclosure, whereby said enclosure can be opened with the application of force on the distal end of the strip.

Preferably, said adhesive bandage is covered by protective tabs which are attached to said upper layer, whereby said tabs are removed when said enclosure is opened.

Preferably, the strips are bound together in a series by means which allow separation of said strips by the application of force at the proximal end of the strip.

Alternatively, the strips are provided individually with said distal end attached to a surface and proximal end unattached.

Preferably the force applied to separate the upper and lower levels of said enclosure is less than the force required to separate the said distal end of the material strip.

Preferably, the packaging unit is contained within a dispenser said dispenser comprising:
an opening allowing at least one packaging unit to be pulled through said opening via said distal tab;
securing means to secure at least one packaging unit once it is pulled outside said dispenser;
whereby an individual packaging unit can be secured once it is outside said dispenser and said enclosure can be opened by pulling on said distal tab and said packaging unit can subsequently be separated by tearing along said perforated edge.

In a second aspect the invention provides a method of manufacturing the packaging unit as described herein.

More specific features for preferred embodiments are set out in the dependent claims.

### OBJECTS OF THE INVENTION

It is an object of the present invention to provide a packaging unit having means to release contents of an enclosure formed by a fold of a material strip and present them for usage by pulling one end of said material strip in a single fluid movement.

It is an object of the present invention to provide a packaging unit providing the means to release its sealed contents using only one hand.

It is a further object of the present invention to provide a packaging unit with a sterile enclosure.

It is a further object of this invention to provide a packaging unit allowing a simple and efficient storage and release of contents.

It is a further object of this invention to provide a packaging unit that releases a bandage which can be applied to a wound using only one hand.

Further objects and advantages of the present invention will be disclosed and become apparent from the following description. Each object is to be read disjunctively with the object of at least providing the public with a useful choice.

### BRIEF DESCRIPTION OF THE DRAWINGS

FIG. 1 shows a perspective and side view of the simple z-fold packaging unit, demonstrating the z-fold aspect which allows a plurality of packaging units to be stacked and the two tabs to protrude in opposite directions.

FIG. 2 shows a perspective and side view of the packaging unit demonstrating the v-folded edge with the sealed enclosure.

FIG. 3 shows a perspective view of a packaging unit in the first stage of opening the enclosure containing an adhesive bandage.

FIG. 4 shows the packaging unit open with protective tabs covering the bandage and its adhesive surfaces.

FIG. 5 shows the packaging unit open with an adhesive bandage which has one of its protective tabs removed.

FIG. 6 shows the packaging unit open with an adhesive bandage which has both of its protective tabs removed.

FIG. 7 shows a perspective view of a plurality of packaging units which are attached to a dispensing container.

FIG. 8 shows a perspective view of a packaging unit after being partially opened.

FIG. 9 shows a perspective view of a packaging unit after being partially opened with part of the adhesive strip exposed.

FIG. 10. shows a perspective view of a packaging unit which is attached to a dispensing container after it has been completely opened and presenting an adhesive bandage.

FIG. 11 shows the first stage of a user applying a material strip to a wound with an adhesive bandage.

FIG. 12. shows the second stage of a user applying a material strip to a wound with an adhesive bandage.

FIG. 13 shows the final stage of a user applying a material strip to a wound with an adhesive bandage.

FIG. 14 shows the dispensing container suspended from a door handle.

FIG. 15 shows the user detaching a single packaging unit from the dispensing container by tearing across the perforation at the distal tab.

FIG. 16 shows a perspective view of an alternative dispensing container where the packaging units are connected end-to-end and dispensed through an opening having means to secure individual packaging units.

FIG. 17 shows a side view of the alternative dispensing container in FIG. 16.

### DETAILED DESCRIPTION OF THE INVENTION

The packaging unit of the present invention may be applied to most forms of sterile medical dressing but can equally be utilized for almost any small object which is sealed between two outer protective strips.

Various embodiments of the present invention are described hereinafter with reference to the figures. It should be noted that the figures are not drawn to scale. It should also be noted that the figures are only intended to facilitate the description of specific embodiments of the invention. In addition, an aspect described in conjunction with a particular embodiment of the present invention is not necessarily limited to that embodiment and can be practiced in any other embodiments of the present invention.

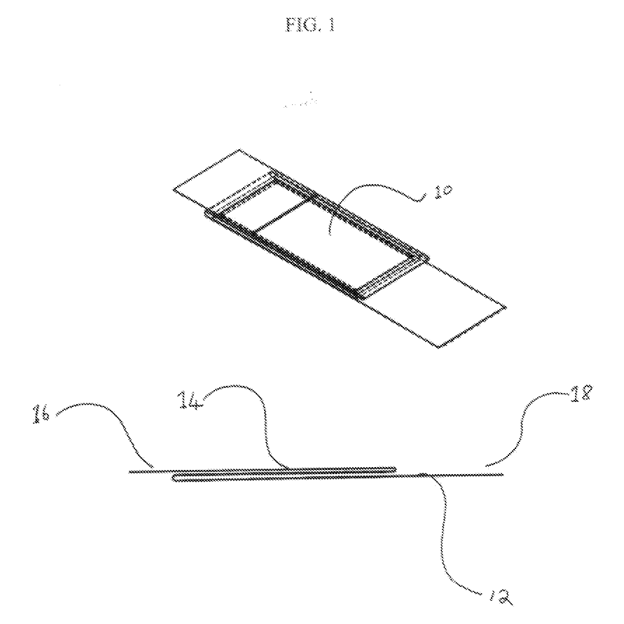

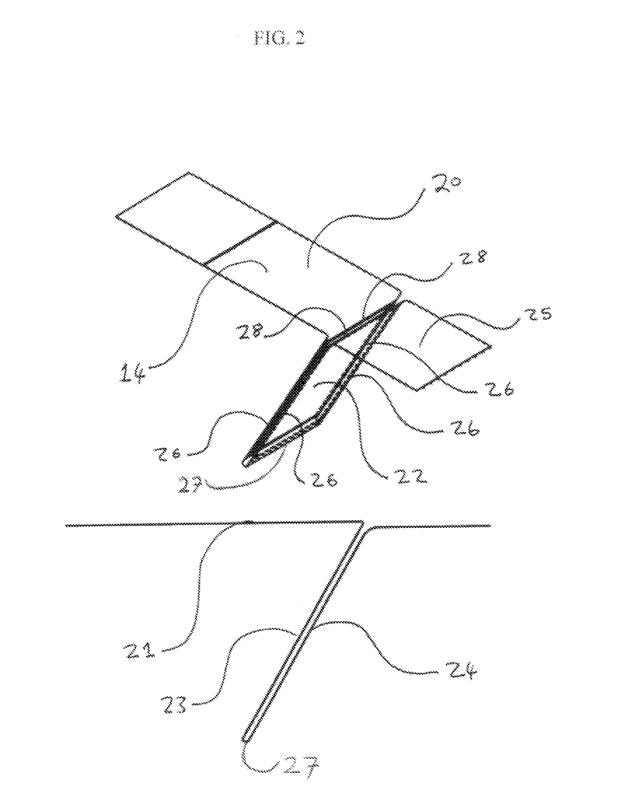

Referring now to the invention in more detail, FIG. 1 shows a top perspective 10 and side view 12 of the simple packaging unit 14, demonstrating the z-fold aspect which allows the distal tab 16 and proximal tab 18 to protrude in opposite directions. FIG. 2 shows a top view 20 and side view 21 of the packaging system ready to be opened with the contents held inside the sealed enclosure 22 of the packaging unit 14. The enclosure is formed between an upper layer 23 and lower layer 24 of the packaging. Although not shown in FIGS. 1 and 2, it will be readily apparent to those skilled in the art that the packaging can be manufactured from a continuous strip of the same material. Preferably, such material may be comprised of plastic (e.g. PVC) or tear resistant paper. In preparing the packaging, the material strip 25 is folded at the v-folded edge 27 around the item to be enclosed and the two side enclosure edges 26 and one top enclosure edge 28 are then sealed with sealing means, such as heat sealing, to form the enclosure 22. Crucially, the distal tab 16 and proximal tab 18 is left protruding from the end of the package 14 which has been sealed, and the proximal tab 18 is folded back onto the upper layer 23 of the enclosure 22 to form the flattened z-fold. However, it will be apparent to those skilled in the art that the distal tab 16 could also be folded back in a similar manner to form an inverted z-fold, in which case reference to the distal and proximal tab is exchanged. This leaves the sealed enclosure 22 in the centre with two tabs pointing in opposite directions ready to be pulled apart to open the packaging. In a single unit, the distal tab 16 is left long enough to protrude beyond the end of the folded and sealed part of the package 14 to provide an easy element to grip.

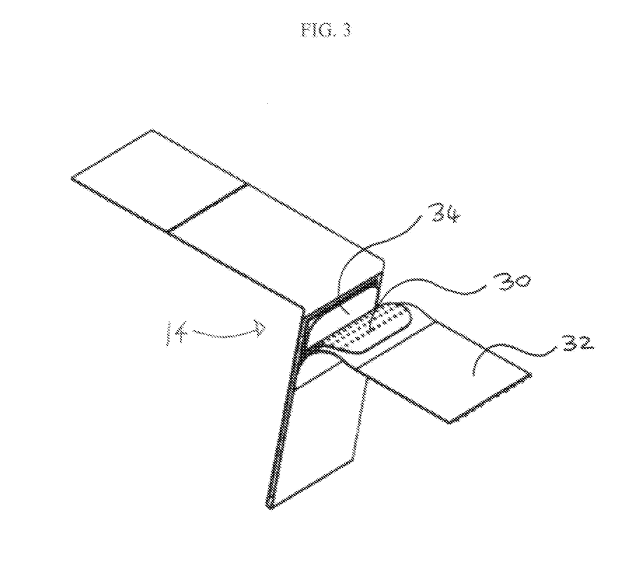

FIG. 3 shows an angled view of a packaging unit 14 in the first stage of opening the enclosure 22 containing an adhesive bandage 30. The material strip is pulled from the proximal tab 32 to open the enclosure. In this particular embodiment, a protective tab 34 covering part of the adhesive bandage is attached to the upper layer 23 of the packaging 14, thus ensuring the adhesive bandage can be applied to a wound.

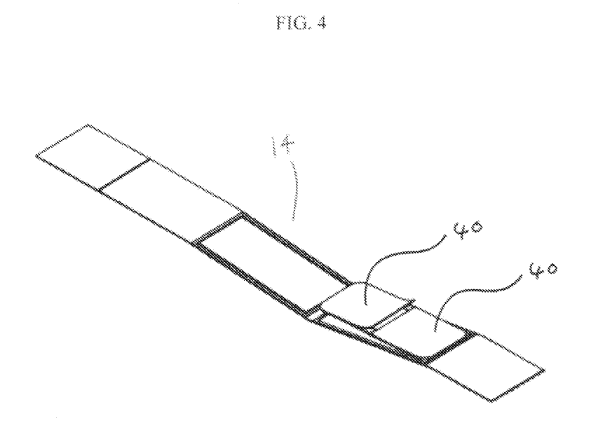

FIG. 4 shows the packaging unit 14 open with protective tabs 40 covering the adhesive bandage and its adhesive surfaces, which are attached to the lower layer of the packaging. In this case, the protective tabs have not been removed when the enclosure is opened.

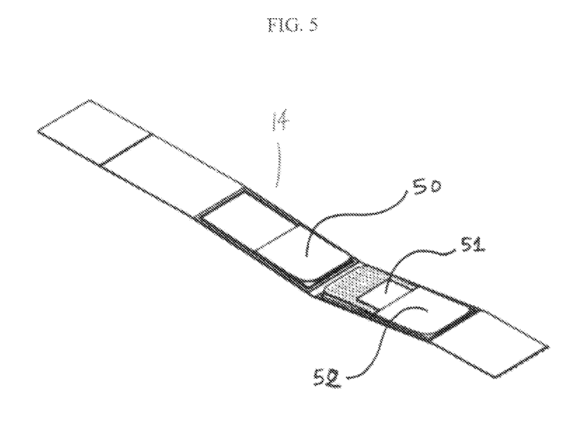

FIG. 5 shows the packaging unit 14 open with an adhesive bandage 51, which has one of its protective tabs 50 removed by being attached to the upper layer and the other protective tab 52 still attached to the adhesive bandage 51. Preferably, the protective tab 50 is attached to the upper layer with adhesive means, such as gum arabic, such adhesive means being stronger than the adhesive force of the protective tab 52 to the adhesive bandage.

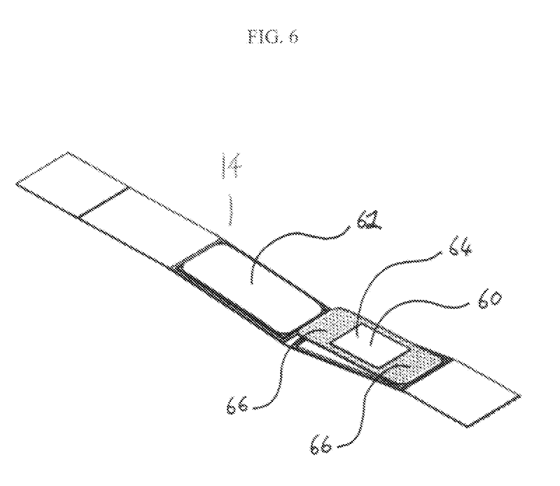

FIG. 6 shows the packaging unit 14 open with an adhesive bandage 60 which has a single protective tab 62 removed, presenting the sterile dressing 64 to be placed over a wound using the adhesive surface 66. An additional significant benefit of this design is that it obviates the necessity for the user to remove the protective tabs with their fingers or even to place their fingers anywhere near the antiseptic dressing and thus greatly reduces the risk of cross-contamination of the dressing with dirt or bacteria before it is applied. Alternatively, there are no protective tabs covering the adhesive bandage, but instead the upper layer 23 of the material strip 25 has means for ensuring the adhesive bandage 51 is kept sterile, such as a silicon layer.

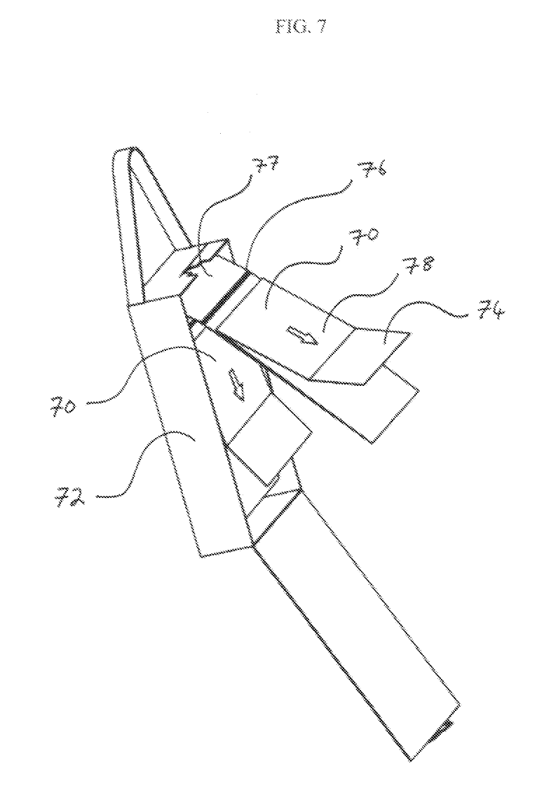

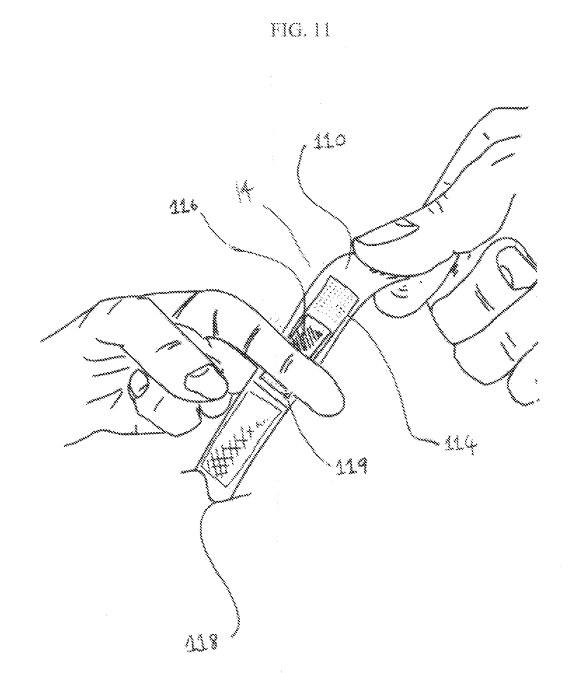

FIG. 7 shows a perspective view of a plurality of packaging units 70 which are attached to a dispensing container 72. The enclosure 22 of the material strip 25 may be opened by pulling on the proximal tab 78. The packaging unit 70 can also be removed by tearing along the perforated edge 76 adjacent to the distal tab 77. In this particular embodiment, proximal tab 78 is longer and has a protruding tab 74 which is folded outwards, which allows it to be gripped more easily by a user. Preferably, the force required to pull on the distal tab 77 to open the enclosure 22 is less than the force required to tear the perforated edge 76. This allows the enclosure 22 to be opened with one hand before removing the packaging unit 70 by tearing the perforated edge 76. Where the enclosure 22 contains an adhesive bandage, this allows the entire packaging unit to be applied to a wound with one hand, as illustrated in the description of FIGS. 11-13 below. Although the dispensing container 72 illustrates a particular embodiment with multiple packaging units 70 dispensed from a stack, it will be apparent to those skilled in the art that different widths, sizes or types of content are easily dispensed alongside each other from a dispensing container 72.

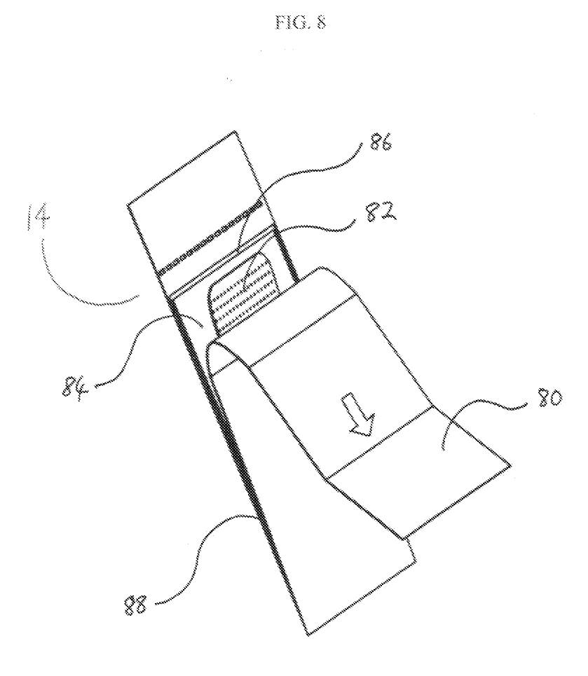

FIG. 8 shows a perspective view of a packaging unit 14 after being partially opened by pulling on the proximal tab 80 which exposes an adhesive bandage 82 contained within the enclosure 84. The adhesive seal around the perimeter has been broken at the top enclosure edge 86 but is still intact at the side enclosure edges 88.

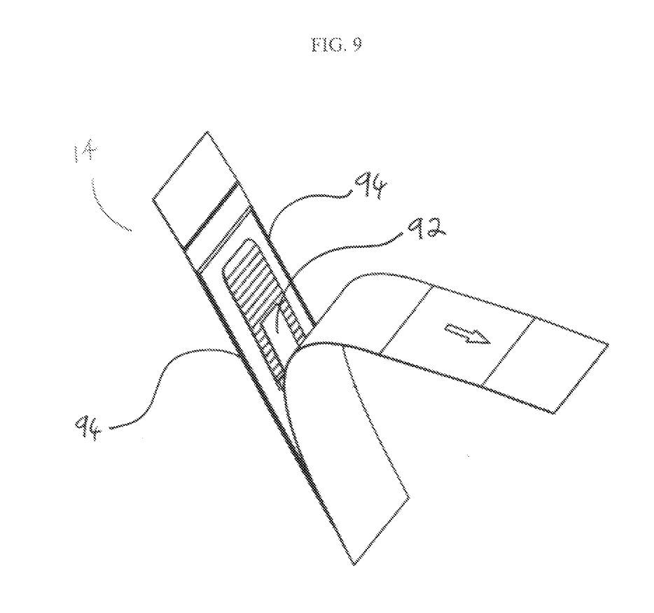

FIG. 9 shows a perspective view of a packaging unit after being opened further with the dressing 92 becoming exposed as the seals are broken along the two outside edges 94.

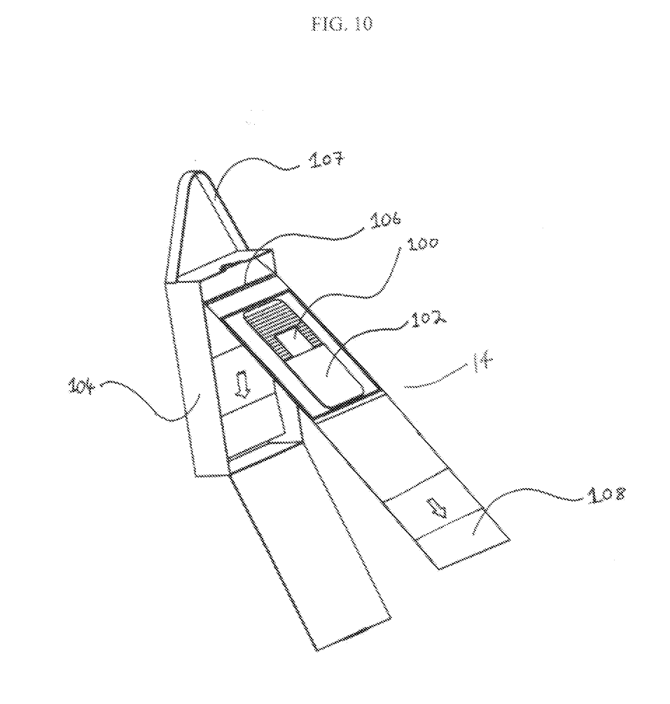

FIG. 10. shows a perspective view of a packaging unit 14 which is attached to a dispensing container 104 after it has been completely opened and presenting an adhesive bandage 100. In this particular embodiment one of the protective tabs 102 is still covering half the adhesive bandage. The material strip can be removed with one hand by tearing along the perforated edge 106 when holding the proximal tab 108. The dispensing container has a loop of material 107 attached to the same end as the distal tabs to enable it to be attached to a door handle, hook, wall or other piece of furniture.

FIGS. 11-13 show the stages in applying an adhesive bandage which has been opened and presented to a wound using the dispenser system described herein, in this case on the index finger of the user's left hand. It should be noted that this being a fairly typical wound creates a clear advantage in being able to open, present and apply an adhesive dressing using only one hand (in this case the right hand).

In FIG. 11, the proximal tab 110 of the packaging unit 14 is held between a finger and thumb of one hand having unfolded and opened up the enclosure. The adhesive surface 119 of the adhesive bandage is exposed and presented facing upwards but still tacked to the lower layer 114 underneath to hold it in place whilst it is being positioned. The adhesive dressing 116 has also been exposed and is presented ready to apply to the wound. The distal tab 118 of the material strip may still be attached to either a stack or continuous roll of packaged plasters which are in turn held in a box which can be hung from a convenient item such as a door handle, thus facilitating the single-handed operation as shown here.

The injured finger in this case is first applied to the proximal end of the adhesive part of the bandage with the wound facing towards the sterile dressing 116 and pressed against the adhesive surface 119.

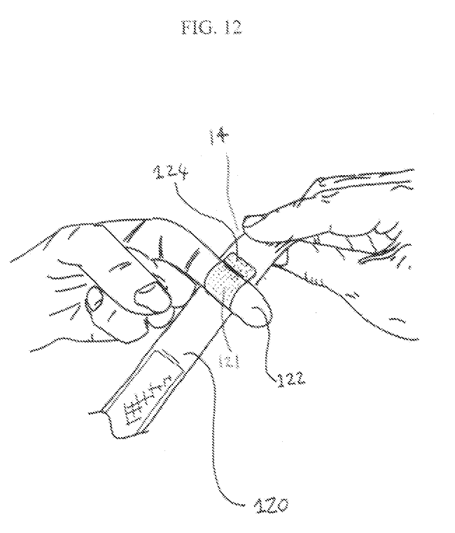

FIG. 12 shows the second stage of a user applying the packaging unit 14 to a wound. By rolling the finger up along the material strip 120, or alternatively simply wrapping the material strip 120 around the finger, the adhesive bandage 121 is simultaneously detached from the packaging unit 14 and adheres to user's finger 122, with the adhesive dressing (not shown) now applied to the wound and the second side of the adhesive surface 124 being pressed up against the finger to secure it in place.

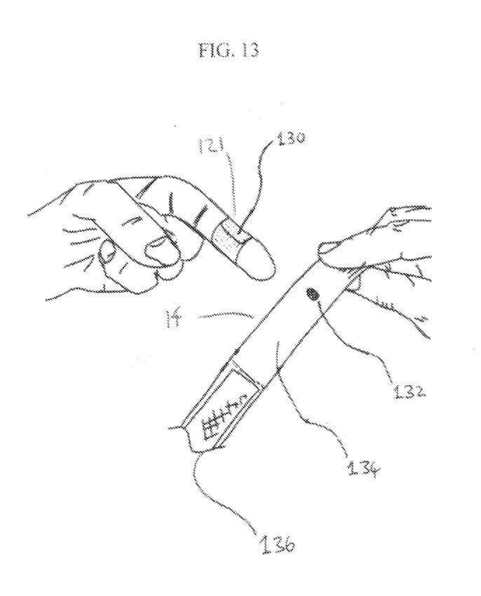

FIG. 13 shows the third and final stage in applying an adhesive bandage 121 which has been opened and presented using the using the system described herein. The adhesive bandage 121 is now neatly applied to the finger 130 having used only one hand and without the necessity of ever touching or getting near the adhesive surfaces or adhesive dressing (except where applied directly to the wound). An adhesive or tacky material 132 (e.g. gum Arabic or similar) which was used to hold the adhesive bandage in position on the material strip 134 while being applied, can be seen still attached to the packaging unit 14. The material strip 134 itself is now ready to be disposed of and if required can be torn from a continuous strip or stack of other items along a perforated edge (not shown) adjacent to the distal tab 136.

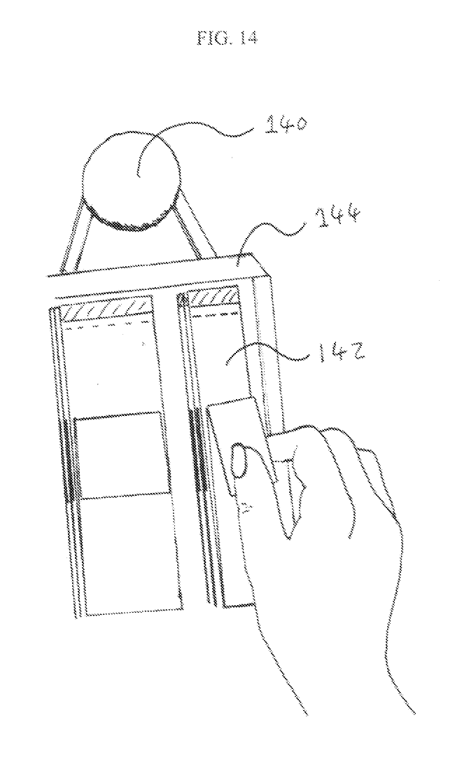

FIG. 14 shows the dispensing container 144 suspended from a door handle 140 with an alternative embodiment of stacked packaging units of different widths 142.

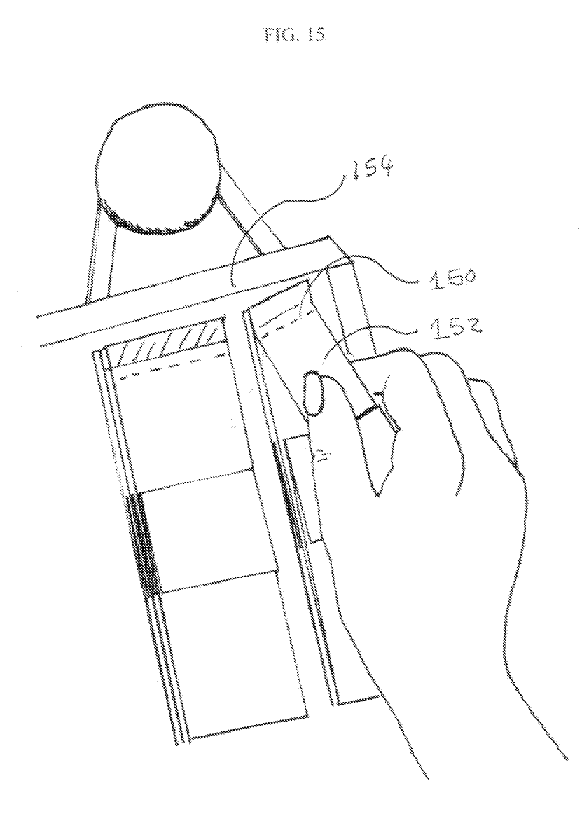

FIG. 15 shows the user detaching a single packaging unit from the dispensing container 154 by tearing across the perforated edge 150 adjacent to the distal tab 152.

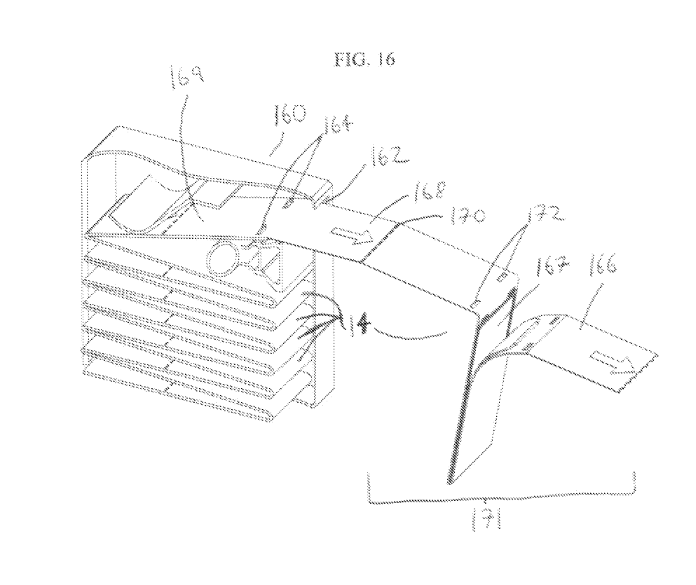

FIG. 16 shows a perspective view of an alternative dispensing container 160 where the packaging units 14 are connected end-to-end and dispensed through an opening 162 having securing means 164 to secure individual packaging units 171. In this embodiment, the proximal tab 166 on the individual packaging unit 171 that has been dispensed from the dispensing container 160 can be pulled to open its enclosure 167. The proximal tab 168 on the adjacent packaging unit 169 is connected end-to-end to the individual packaging unit 171 via a perforated edge 170 which can be torn to remove the individual packaging unit 171. After the individual packaging unit 171 is removed, the proximal tab 169 of the adjacent packaging unit 169 will remain outside the dispensing container 160 which allows a user to pull on it to dispense the adjacent packaging unit 169. It will be apparent to those skilled in the art that subsequent packaging units 14 can be dispensed in a similar manner and the securing means 164 can be configured to allow packaging units 14 to be released from the opening 162 and individually secured. In this embodiment, the means to secure each packaging unit 14 is facilitated using securing holes 172 in the material strip which allows the securing means 164 to secure an individual packaging unit 171 in place, for example, the securing means comprises a spring-loaded mechanism to push a rigid but retractable block through the securing holes 172. Once secured, the proximal tab 166 can be pulled to release the contents of the enclosure 167.

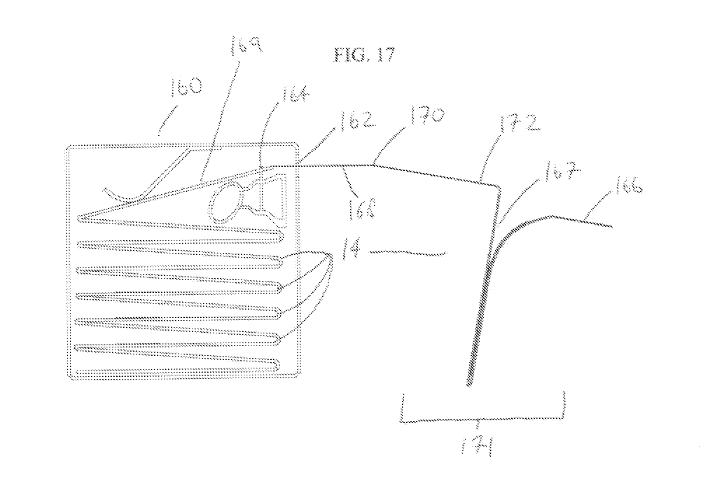

FIG. 17 shows a side view of an alternative dispensing container shown in FIG. 16 with identical reference numbers.

It will be apparent to those skilled in the art that various items could be contained within the enclosure of the packaging unit disclosed herein and/or could be applied to a dispensing system as disclosed herein. These include, but are not limited to:
Chewing gum;
Hypodermic needles;
Tablets;
Disposable contact lenses.
Prophylactics;
Cutlery;
Surgical instruments;
Medical requisites;
Veterinary supplies;
Catering supplies;
Horticultural consumables;
Blades;
Babycare;
Cosmetics.

While the invention has been illustrated and described in detail in the foregoing description, such illustration and description are to be considered illustrative or exemplary and non-restrictive; the invention is thus not limited to the disclosed embodiments. Features mentioned in connection with one embodiment described herein may also be advantageous as features of another embodiment described herein without explicitly showing these features. Variations to the disclosed embodiments can be understood and effected by those skilled in the art and practicing the claimed invention, from a study of the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures can not be used to advantage.

1. A packaging unit comprising:
a material strip folded to form an enclosure, such enclosure being sealed substantially along the edges of the material strip to form an upper and lower layer of said enclosure wherein said upper and lower layers can be separated upon the application of pulling force on either end of the material strip adjacent to said enclosure.

2. A packaging unit claimed in claim 1 further comprising:
material strip having a v-folded edge substantially in its centre to form an enclosure, and said upper and lower layers being substantially parallel to each other;
proximal and distal tabs of material strip which are formed by folding back said upper and lower layers at the top enclosure edge of said enclosure, respectively;
wherein said enclosure is sealed along said top enclosure edge and its side enclosure edges;
wherein said upper and lower layers of said enclosure can be separated by pulling on the proximal and/or distal tab of said material strip;
whereby said enclosure is opened to dispense its contents.

3. A packaging unit claimed in claim 1 wherein the contents of said enclosure is an adhesive bandage although any other item having characteristics which allow stacked arrangement of the packaging units can be substituted.

4. A packaging unit claimed in claim 2, wherein the proximal end of said material strip is secured to an adjacent packaging unit with a strength greater than the adhesive strength of the inner edges of said enclosure, whereby said enclosure can be opened with the application of pulling force on the distal end of the strip.

5. A packaging unit claimed in claim 3, wherein said adhesive bandage is covered by protective tabs which are attached to said upper layer, whereby said tabs are removed when said enclosure is opened.

6. A packaging unit claimed in claim 1 wherein said packaging units are bound together in a series by means which allow separation of said strips by the application of force at the proximal end of said material strip.

7. A packaging unit claimed in claim 1 wherein said packaging units are provided individually with said distal end attached to a surface and proximal end unattached.

8. A packaging unit claimed in claim 7 wherein the force applied to separate the upper and lower levels of said enclosure is less than the force required to separate the said distal end of the material strip.

9. A packaging unit claimed in claim 6 contained within a dispenser said dispenser comprising:
an opening allowing at least one packaging unit to be pulled through said opening via said distal tab;
securing means to secure at least one packaging unit once it is pulled outside said dispenser;
whereby an individual packaging unit can be secured once it is outside said dispenser and said enclosure can be opened by pulling on said distal tab and said packaging unit can subsequently be separated by tearing along a perforated edge.
